(19) United States
(12) Patent Application Publication
(10) Pub. No.: US 2009/0079936 A1
(43) Pub. Date: Mar. 26, 2009

(54) DEVICE FOR TAKING PHOTOGRAPHS OF THE FUNDUS OF THE EYE (FUNDUS OCULI)

(75) Inventor: Yves Robert, Zurich (CH)

Correspondence Address: RANKIN, HILL & CLARK LLP, 38210 Glenn Avenue, WILLOUGHBY, OH, 44094-7808, US

(73) Assignee: OPHTHAMETRICS AG, Altendorf (CH)

(21) Appl. No.: 12/282,810

(22) PCT Filed: Mar. 13, 2006
(86) PCT No.: PCT/CH2006/000147

§ 371 Date: Oct. 10, 2008

(51) Int. Cl. A61B 3/14; A61B 3/15; A61B 3/12

(52) U.S. Cl. 351/206; 351/246

(57) ABSTRACT

The device is used for taking photographs of the fundus of the eye (fundus oculi). It comprises: A) an illumination source (1); B) illumination optics (1; 2; 4; 8; 9; 10), which generate light in at least two different frequency bands and which can sequentially direct the light onto the fundus; C) focusing optics (2; 3; 14; 15; 16), which can sequentially generate images of the fundus that is illuminated with different frequency bands; and D) a photographic camera (3), which can record the generated images. The device makes it possible, with minimal illumination intensity and without medicinally induced dilation of the patient's pupils, to generate high-quality colour images and, moreover, to record spectrally resolved images. The device also makes it possible to determine different blood values, particularly the haemoglobin concentration, and the opacity of the crystalline lens.

The invention refers to a device for a photographic mapping of the eye ground (fundus oculi) according to the main concept of patent claim 1.

According to the state of the art, the mapping of the eye ground employs either white light (flashlight) or narrow-band light of a few frequency bands irradiated simultaneously. The spectral break-down occurs—if required by the measuring process—only in the mapping optics at the time of mapping the image. Because the lighting employs light of various frequency bands simultaneously or over the entire visible spectrum, the instantaneous intensity of the light must be selected at a commensurately high value, which triggers the known pupillary reflex. In order to eliminate this undesirable effect, the patient's pupil first needs to be dilated by medication.

The invention aims at remedying this. The purpose underlying the invention is to provide a device (an eye ground camera) that allows mapping, at low lighting intensity and without a medicated widening of the patient's pupil, of high quality colour images on one hand and of spectrally resolved images on the other hand. Beyond this, the device according to the invention (eye ground camera) should enable assessing various blood parameters, notably haemoglobin concentration and eye lens opacity.

The invention solves this task through a device presenting the characteristics of claim 1.

The device according to the invention is remarkable in that in lighting the eye ground it employs a narrow-band light (of preferably low intensity) made up of several, but at least two frequency bands applied successively in time, while memorizing an image of the eye ground in each spectral range. Thanks to the slight instantaneous lighting intensity, the pupillary reflex is largely avoided. The patient fails to be blinded, thus allowing the measurement to be repeated after a brief time period.

A characteristic of the invention is the fact that the spectral break-up already occurs when lighting the eye ground, and that the individual frequency bands are applied at separate times. This allows keeping the light's instantaneous intensity at a low level. If the light's colour range is crossed from one end of the visible spectrum to the other while registering the eye ground images of the various frequency bands, these can later be assembled into an overall colour image in practice closely approaching that of a mapping under white light. A spectral break-up of the light is essential both for determining blood parameters and measuring eye lens opacity, and is in this case already available based on the lighting method. Red light is of prime interest in defining haemoglobin concentration, because of its preferential reflection by the blood. Blue light is on the other hand advantageous in determining eye lens opacity: this process exploits the contrast between fovea and pupil which occurs best in the blue colour range.

Another characteristic of the invention is the fact that the spectral break-up of the lighting action in succession allows using a black and white CCD camera for image mapping. From a technical viewpoint, black and white CCDs are considerably more sensitive than colour CCDs.

To use the inventive device for mapping the eye ground at an undilated pupil, it should be functionally capable of operating at a pupillary diameter of a maximum 3-4 mm. Eye motions relative to the mapping camera limit the useful aperture to about 2 mm. Both the lighting and mapping must be performed through this opening. Because of the internal reflections, especially of the cornea and the eye lens itself, the lighting and the mapping beams must be separated in space. For this purpose, the available aperture is concentrically divided, into an outer ring for lighting and an inner ring for mapping. The mapping circle should be as large as possible, as this allows maximizing both diffraction-restricted resolving capacity and lighting efficiency. The concentric lighting ring should on the other hand not be chosen too thin, so as to prevent an excess lighting intensity when applying a certain power. A preferred selection is to provide the same lighting and mapping areas, or at any rate a slightly larger central mapping area.

The mapping camera may be a black and white camera.

In a special form of embodiment, the eye ground camera avails itself of a positioning device, which helps to make it possible to adjust the eye's and the mapping camera's optical axis as well as the distance between the mapping optics (2;3;14;15;16) and the cornea in a precise and reproducible manner. The U.S. Pat. No. 5,474,451 (by Robert et al.) describes this process. On one hand, the reproducibility of the position is important in order to promptly adjust for the unavoidable reflections and losses while taking absolute blood parameter measurements. On the other hand, the reproducibility is important for the successive mapping of eye ground images in various spectral ranges, so as to picture identical sections as sharply as possible, in order to enable their later reassembling into a single colour image at minimum computing effort. The eye ground camera's optical design is such as to make its lighting efficiency as large as possible at the mentioned marginal conditions.

The frequency bands may offer a respective band width of 5-50 nm, preferably of 10-30 nm.

The light projected onto the eye ground may be within the visible range (preferably at a wave length between 400 and 800 nm) or in the infra-red range.

In a particular form of embodiment the generated images can be memorized in digital form.

The time intervals between the individual frequency band should be appropriately kept as short as possible, preferably shorter than 100 ms and typically shorter than 20 ms.

In order to allow generating an overall eye ground image from the mappings achieved by the inventive device, the computer employed is preferably one allowing the individual images to be digitally superimposed.

The inventive device allows performing the following process for a photographic mapping of the eye ground (fundus oculi):
a) A lighting of the fundus occurring in a time succession, while using a localized light comprising at least two different frequency bands,
b) Photographic mappings of the eye ground, while successively lighted with various frequency bands using a photographic image mapping camera.

The photographic eye ground mappings obtained in a time succession are preferably superimposed to create an overall image (colour image).

In a particular form of embodiment of this process the overall power irradiated into the eye amounts to 30-100 μW. The eye ground's time exposure at each individual frequency band is preferably in the range of 10-30 ms.

The invention and its further developments will be described in further detail and with the aid of partially simplified representation on the following example of embodiment:

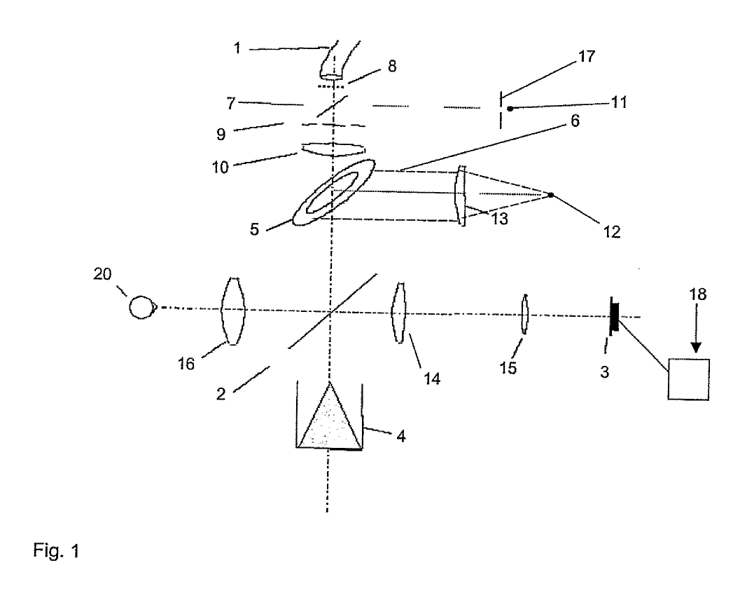

FIG. 1 shows the simplified layout of a device according to the invention.

The device according to the invention consists essentially of a lighting source (1), a mapping optics and a lighting optics. Both radiating beams are coupled by a first beam splitter 2. The first beam splitter 2 is chosen so that about 95% of the light reflected from the eye ground falls on the image mapping camera 3. In correspondence, only about 5% of the lighting power reaches the eye, but without posing a problem, as adequately strong lighting sources are available. The light crossing the first beam splitter 2 in a straight line is almost totally absorbed by a beam absorber 4. In addition to the path of the lighting beam, a positioning beam 6 according to U.S. Pat. No. 5,474,451 (Robert) is coupled-in through a ring-shaped mirror 5. A second beam splitter 7 also provides a light point to guide the patient's eye.

The function of the individual components of the device will be described according to FIG. 1 as follows:

### Lighting Source:

The necessary light is for instance coupled-in from a cold light source over a fibre bundle (top of FIG. 1). An IR-LED device can alternatively be installed to produce infrared images.

### Lighting Optics (1;2;4;8;9;10):

A tunable or mechanically fast changeable interference filter 8 generates a narrow band light, with a typical band width of 10 to 30 nm.

A diaphragm 9 with a central beam stop can prevent the lighting of the central portion of the cornea, so as to avoid generating any reflex effect in the image mapping camera 3. The lighting beam lens 10 provides for a uniform illumination of the eye ground. The first beam splitter 2 reflects about 5% of the light in the direction of the eye 20, the transmitted portion is rendered harmless by the beam absorber 4.

The second beam splitter 7, which is set up beneath the lighting source 1 in the form of a lighting beam bundle, serves to focus-in a tiny light point. This light point is generated by a LED 11 with a downstream pinhole 17. The patient directs his eye to the light point, thus allowing the eyeball to assume a defined position.

If the eye ground camera is properly set up, the IR-lighting 12 for the patient's eye-positioning, together with the gathering lens 13 and the ring-shaped mirror 5, generates a converging light beam which is reflected from the cornea in parallel (Patent Robert et al.). The ring-shaped mirror 5 is shown in FIG. 1 as an elliptic mirror with an elliptic hole in the centre. The main axes of the ellipse are chosen so as to make the mirror, in a 45° projection, appear like a circle with a round hole.

### Image Mapping Optics (2;3; 14; 15; 16):

An atmospheric ophtalmoscopic lens 16 is used to map the eye ground. 95% of the light reflected by the eye ground passes the first beam splitter and is projected, by the first mapping lens 14 and the second mapping lens 15, in a magnified form on the CCD-Chip.

### Image Mapping Camera:

The image mapping camera 3 is used is a CCD camera connected over an interface to a PC, so as to enable the mapped image to be digitalized, memorized and further processed.

The beam path is shielded from extraneous and stray light by a blackened housing.

### Positioning of the Device:

A cross-table allows the device to be finely adjusted with respect to the patient's eye. The adjusting can in this case by done by hand or by evaluating the mentioned positioning light bundle 6 and appropriate actuators in an automatic manner.

1: A device for the photographic mapping of the eye ground (fundus oculi) comprising:
A) a lighting source;
B) a lighting optics capable of generating light of at least two different frequency bands, and of successively directing it on the eye ground;
C) an imaging optics capable of successively generating images of the eye ground lighted with different frequency bands; and
D) a photographic image mapping camera capable of recording the generated images.

2: The device according to claim 1, wherein the image mapping camera is a black and white CCD camera.

3: The device according to claim 1, further comprising a positioning device through which the eye's optical axis and the image mapping camera, as well as the distance between the imaging optics and the cornea can be adjusted.

4: The device according to claim 1, wherein the frequency bands each have a band width of 5-50 nm.

5: The device according to claim 1, wherein the light directed on the eye ground is in the visible range.

6: The device according to claim 1, wherein the light directed on the eye ground is in the infrared range.

7: The device according to claim 1, wherein the generated images can be memorized in digital form.

8: The device according to claim 1, wherein the time intervals between the individual frequency bands are smaller than 100 ms.

9: A device to generate an overall image of the eye ground from the images obtained by the device according to claim 1, wherein it comprises a computer whereby the individual images can be digitally superimposed.

10: A process for the photographic mapping of the eye ground (fundus oculi), comprising the steps:
a) successively lighting the eye ground with a spectrally delimited light comprising at least two different frequency bands, and
b) photographic mapping of the eye ground successively lighted at various frequency band using a photographic image mapping camera.

11: The process according to claim 10, wherein the successively obtained photographic mappings of the eye ground are superimposed to an overall image (color image).

12: The process according to claim 10, wherein the eye's optical axis and the image mapping camera as well as the distance between the image mapping camera and the cornea can be reproducibly adjusted.

13: The process according to claim 10, wherein the total power radiated into the eye amounts to 30-100 μW.

14: The process according to claim 10, wherein the light directed on the eye ground is in the visible range.

15: The process according to claim 10, wherein the light directed on the eye ground is in the infrared range.

16: The process according to claim 10, wherein the duration of the eye ground exposure at each of the frequency bands is in the range of 10-30 ms.

17: Application of the device according to claim 1, for determining the degree of opacity of the eye lens.

18: Application of the device according to claim 1, for determining blood parameters.
